(19) United States
(12) Patent Application Publication
(10) Pub. No.: US 2013/0261622 A1
(43) Pub. Date: Oct. 3, 2013

(54) IMPLANT FOR PROXIMAL FRACTURES OF LONG BONES

(71) Applicants: CHIRMAT SARL, Monthey (CH); UNIVERSITE DE SAVOIE, Chambery (FR); HEPIA, Geneva (CH); TURAL, Marignier (FR)

(72) Inventors: Christian BONJOUR, Begnins (CH); Yvan ARLETTAZ, Monthey (CH); Pierre VACHER, Thorens-Glieres (FR); Franck TOUSSAINT, Annecy (FR); Remi BILLARD, Raphael (CH); Laure DEVUN, Saint-Jerome (FR); Thomas GRADEL, Marignier (FR); Eric VITTECOQ, Groisy (FR)

(73) Assignees: Chirmat Sarl, Monthey (CH); Tural, Marignier (FR); Hepia, Geneva (CH); Universite de Savoie, Chambery (FR)

(21) Appl. No.: 13/828,178

(22) Filed: Mar. 14, 2013

(30) Foreign Application Priority Data
Mar. 28, 2012 (CH) 2012CH-0436
May 24, 2012 (CH) 2012CH-00725

(51) Int. Cl. A61B 17/72

(52) U.S. Cl. CPC A61B 17/7233
USPC 606/64

(57) ABSTRACT

Surgical implant for treating fractures of long bones, notably of the femur, having an intramedullary section (52) of elongated shape, adapted for being inserted into the medullary cavity (37), an extramedullary section (56) substantially parallel to the intramedullary section (52), a deformable curved loop (57) connecting the intramedullary section (52) to the extramedullary section (56), an inclined screw (61) traversing said intramedullary section (52) and said extramedullary section (56). By deforming the loop (57), a continuous adjustment of the angle (α) of the cephalic screw (61) and thus an optimal adaptation of the implant to the individual anatomy can be achieved. Thanks to its geometry, this implant induces a protection of the greater trochanter and limits the mechanical loads between the cephalic screw (61) and the rest of the implant.

### REFERENCE DATA

The present application claims priority from Swiss patent application 00725/12 of May 24, 2012 and from Swiss patent application 00436/12 of Mar. 28, 2012, both incorporated hereby by reference in their entirety.

### TECHNICAL FIELD

The present invention relates to an implantable device for treating proximal fractures of long bones. Notably, but not exclusively, it relates to an implant that can be used for treating pertrochanteric femoral fractures. The present invention also addresses fractures of the proximal humerus or of other long bones having similar problems and therapies.

### STATE OF THE ART

The technique presented hereafter relates to fractures of the proximal femur; techniques for treating fractures of all long bones are similar.

Pertrochanteric femoral fractures are fractures relating to the proximal part of the femur close to the hip joint. These traumata belong to fractures called in everyday language femoral neck fracture and most often affect the elderly. Their therapy is based, in the majority of cases, on a quick surgical treatment with anatomically correct reduction followed by the placing of an implant that stabilizes the fracture. It has been demonstrated that these procedures allow gait reeducation within relatively short times and improve considerably the recovery rate.

The techniques currently used include stabilizing the facture either by means of intramedullary implants inserted in the diaphyseal cavity or by plates or blades screwed onto the bone. In all cases, one or several so-called cephalic screws or other devices oriented along the axis of the femoral neck are used to stabilize the head of the femur.

The device disclosed by EP0546460B1, for example, comprises a trochanter-stabilizing plate that can be connected by a screw to a centromedullary nail and comprising two inclined screws, on either side of the medullary cavity, for stabilizing the head and neck of the femur. On the other hand, U.S. Pat. No. 7,632,272 discloses an intramedullary fastening device with an inclined screw designed to insert into the head of the femur and a plate for stabilizing the greater trochanter.

The angle of the femur neck relative to the femoral shaft is greatly variable relative to individual morphology, gender and age. It is thus necessary for implants to be available that reconstruct the angulation that is anatomically correct for each patient. This currently leads to a multiplication of the number of implants that need to be supplied to each hospital to allow emergency treatment of this type of fracture.

Furthermore, so-called intertrochanteric or pertrochanteric fractures often cause a rupture of the upper part of the femur called “great trochanter” to which are attached several muscles responsible for stability and gait that exert there considerable forces. The implants generally used do not always provide adequate stabilization of fractures of the great trochanter.

### BRIEF SUMMARY OF THE INVENTION

One aim of the present invention is to propose an implant that is exempt from the limitations of the known devices and that is notably easily adaptable to a plurality of anatomical configurations so as to reduce the number of reference implants in hospitals.

Another aim of the invention is to propose an implant that, by its very geometry, provides efficient protection to the great trochanter in intertrochanteric or pertrochanteric fractures and limits the mechanical loads between the cephalic screw 61 and the rest of the implant.

According to the invention, these aims are achieved by means of the object of the attached claims and notably by a surgical implant having an intramedullary section of elongated shape, adapted for being inserted into the medullary cavity of a fractured long bone, an extramedullary section substantially parallel to the intramedullary section, a curved loop that can be deformed to adapt to the anatomy of the patient and connecting the intramedullary section to the extramedullary section, an inclined cephalic screw traversing said intramedullary section and said extramedullary section.

### BRIEF DESCRIPTION OF THE FIGURES

An example of embodiment of such an implant is illustrated by FIGS. 1a-1c.

Other variant embodiments of the invention are given in the description illustrated by FIGS. 2a-2c.

FIG. 3 shows, in a simplified manner, an implant according to one aspect of the invention in position in the proximal part of a femur, in a frontal cross-section.

The identical characteristics presented in several figures are indicated by the same reference sign. The same reference sign is used in some cases to indicate locking and fastening screws or their axis in a simplified representation.

### Example(s) of Embodiments of the Invention

The embodiments presented refer only to an implant for fractures of the human femur. The present invention however also includes specific variants for treating fractures of other long bones, notably of the humerus. This invention can also apply in the field of veterinary medicine.

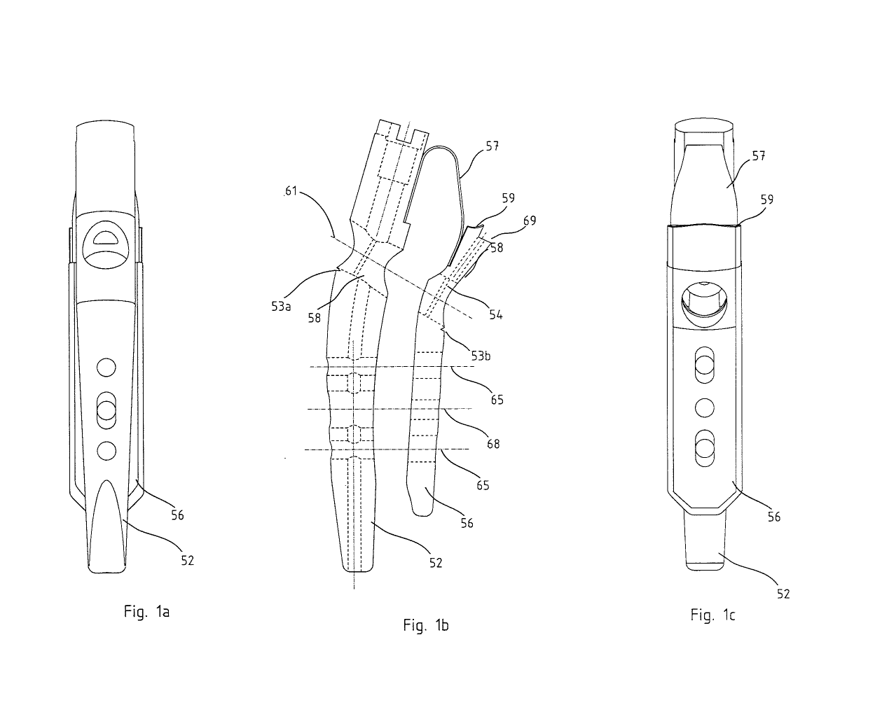

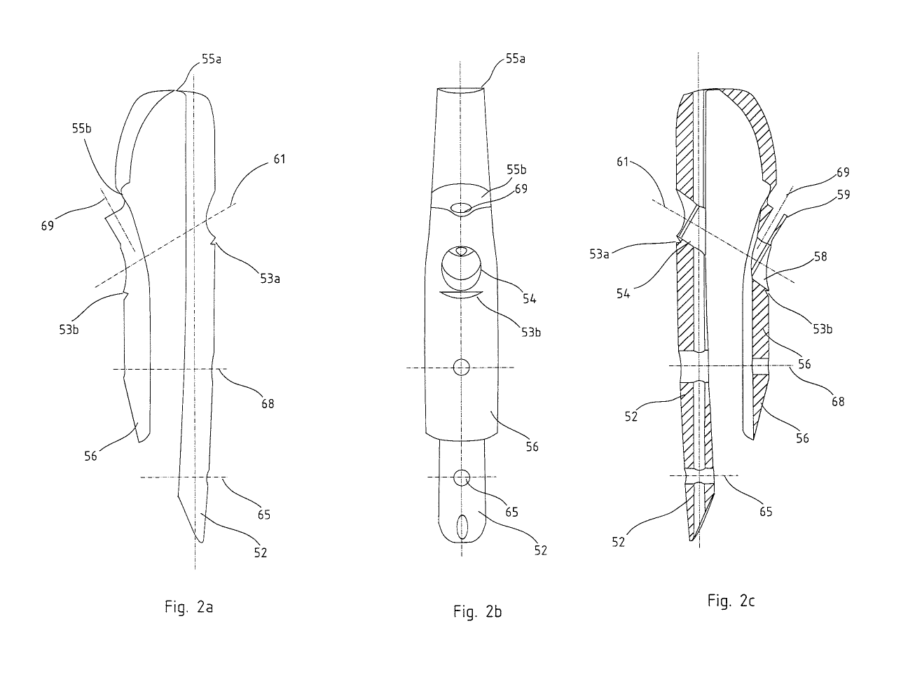

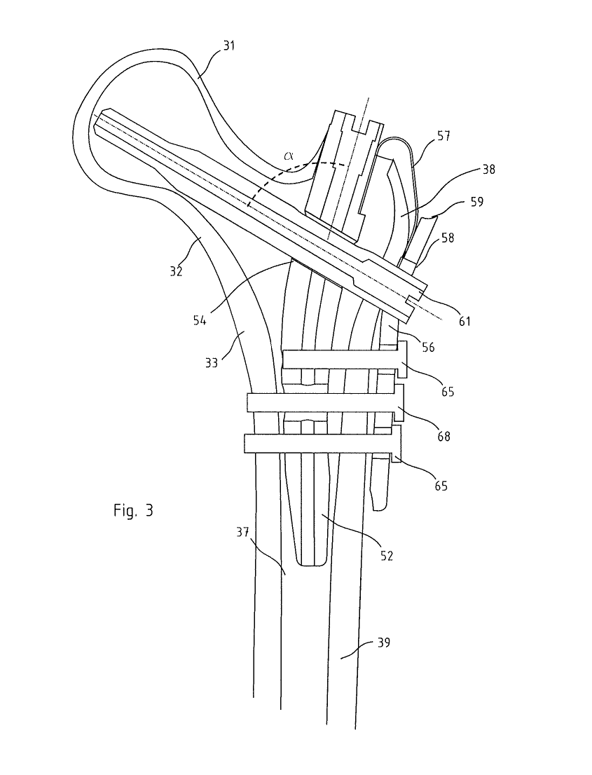

FIGS. 1a-1c illustrate one element of the implant according to one aspect of the invention and comprising an intramedullary section 52 of elongated shape (straight or curved) and adapted for insertion into the medullary cavity 37 (visible in FIG. 3) of the bone in question, for example the femur. The implant also comprises an extramedullary section 56 substantially parallel to the intramedullary section 52.

The curved loop 57 connecting the intramedullary section 52 to the extramedullary section 56 has a shape adapted to match the anatomic profile of the greater trochanter. Advantageously, the loop 57 can be deformed in order for the implant to adapt to the individual morphology of each patient and make easier the insertion of the implant into the bone.

The deformation of the loop 57 can be achieved in several ways. In one variant embodiment, represented schematically in FIG. 1, the loop 57 is constituted by a blade that can be elastically deformed or not thanks to its relatively reduced thickness. According to another variant embodiment, illustrated in FIGS. 2a-2c, the loop 57 comprises a certain number of segments of reduced section (55a-55d) forming deformable hinges. Thanks to this characteristic, the surgeon can deform the implant and achieve the desired degree of curvature by applying a reasonable force.

FIG. 3 shows diagrammatically the inventive implant in place. The intramedullary section 52 inserted into the diaphyseal cavity 37 of a femur 39 can be seen, whilst the extramedullary section 56 rests on a lateral side of the bone and the loop 57 surrounds and protects the greater trochanter 38. The diaphyseal cavity 37 will previously have been opened and bored to the desired diameter using known techniques.

The inventive implant also comprises an inclined cephalic screw 61 traversing the intramedullary section 52 and the extramedullary section 56 through two openings 54 resp. 58. The angle a between the screw 61 and the intramedullary section 52 corresponds to the inclination of the femur neck, so that the screw 61 can traverse the neck 32 along its axis, penetrate into the femur head 31 and make it integrally united.

In a significant manner, the angle a of the screw 61 can be modified by acting on the curvature of the loop 57 and the alignment of the openings 58, 54 in order to best adapt to the anatomy of each patient. The ideal angle a can be determined by preoperative radiographs of the healthy contralateral hip or prior to the implantation, once the fracture has been reduced, thanks to a surgical radioscopy device. The implant is pre-adjusted prior to being inserted into the femur and a possible re-adjustment once the implant is in place can be achieved by pressing on the excrescence 59. Two notches 53a and 53b allow the axis of the cephalic screw 61 to be made visible by radioscopy prior to its introduction, so as to determine whether a re-adjustment is necessary or not. The shape of the openings 54 and 58 is adapted to this relative movement between the intramedullary part 52 and the extramedullary part 56.

The cephalic screw 61, after being positioned, is preferably locked to prevent it from rotating relative to the intramedullary part 52 and/or relative to the extramedullary part 56, as shown in FIG. 2c, by means of the screw 69. The mechanical loads generated by the cephalic screw onto the rest of the implant are thus reduced as compared with implants of conventional type.

Several fastening means are conceivable for holding into place the intramedullary and extramedullary parts of the implant.

With respect to the intramedullary part, one or several locking screws 65 are screwed into the cortical bone. For the extramedullary part, one or several screws 68 are either screwed into the cortical bone or into the intramedullary part of the implant.

The through-holes for the screws 61, 65, 68 in the intramedullary and extramedullary parts 56, 52 can be circular or oblong.

The intramedullary section 52, la extramedullary section 56 and the loop 57 are preferably constituted by a single solid element, for example a metallic element made of titanium or in a biocompatible alloy or any other biocompatible material. The implant of the invention thus includes a very reduced number of detachable parts. This monobloc implant can however be manufactured in several parts that are then assembled (by welding for example).

### REFERENCE NUMBERS USED IN THE FIGURES

31 femur head
32 femur neck
33 fracture
37 diaphyseal cavity
38 greater trochanter
39 diaphysis
52 intramedullary section
53 control notch of the cephalic screw angulation
54 intramedullary guiding cephalic screw
55a-d hinge
56 extramedullary section
57 loop
58 extramedullary guiding cephalic screw
59 resting zone of the angle adjustment
61 cephalic screw
65 locking screw intramedullary part
68 locking screw extramedullary part
69 locking screw preventing the rotation of the cephalic screw
α angle

1. Surgical implant for treating fractures of long bones, having an intramedullary section of elongated shape, adapted for being inserted into the medullary cavity of a fractured long bone, an extramedullary section substantially parallel to the intramedullary section, a curved loop connecting the intramedullary section to the extramedullary section, an inclined screw traversing said intramedullary section and said extramedullary section, characterized in that said loop can be deformed to adapt to the anatomy of the patient.

2. The implant of claim 1, wherein said curved loop is adapted for surrounding and protecting the greater trochanter when the implant is in place.

3. The implant of claim 1, wherein said loop comprises a blade that can be elastically deformed or not by applying a force prior to or during the implantation.

4. The implant of claim 1, comprising means for blocking the rotation of said inclined screw.

5. The implant of claim 1, wherein said intramedullary section, said extramedullary section and said loop are constituted by an assembly of several elements.

6. The implant of claim 1, wherein said intramedullary section, said extramedullary section and said loop are constituted by a single monobloc element.

7. The implant of claim 1, wherein said extramedullary section comprises one or several openings enabling it to be fastened onto one side of said bone by screws or any other fastening means.

8. The implant of claim 1, wherein said loop has segments of reduced section (55a-55d) enabling it to be deformed by an applied force.
